(19) United States
(12) Patent Application Publication
(10) Pub. No.: US 2024/0044923 A1
(43) Pub. Date: Feb. 8, 2024

(54) METHOD OF SELECTING A GILT FOR BREEDING

(71) Applicant: Purdue Research Foundation, West Lafayette, IN (US)

(72) Inventors: Theresa M. Anderson, East Middlebury, VT (US); Christina R. FERREIRA, West Lafayette, IN (US); Kara STEWART, West Lafayette, IN (US); Kayla MILLS, Columbia, MD (US)

(73) Assignee: Purdue Research Foundation, West Lafayette, IN (US)

(21) Appl. No.: 18/258,489

(22) PCT Filed: Dec. 17, 2021
(86) PCT No.: PCT/US2021/064114

§ 371 Date: Jun. 20, 2023

Related U.S. Application Data
(60) Provisional application No. 63/129,184, filed on Dec. 22, 2020.

(51) Int. Cl. G01N 33/92

(52) U.S. Cl. CPC G01N 33/92; G01N 2570/00

(57) ABSTRACT

A method of eliminating a gilt from selection for breeding comprising (i) obtaining a vaginal lipidome profile from a gilt at around the time of weaning and (ii) using the vaginal lipidome profile to assign the gilt as probable fertile or infertile; a method of analyzing a lipidome profile of a gilt vaginal swab comprising (i) receiving a vaginal swab obtained from a gilt at around the time of weaning and (ii) analyzing, or having analyzed, lipids obtained from the vaginal swab, and (iii) determining, or having determined, decreases/increases in lipid levels compared to normal; and a kit for obtaining a vaginal swab from a gilt.

### CROSS REFERENCE TO RELATED APPLICATIONS

This application claims priority to U.S. provisional patent application No. 63/129,184, which was filed Dec. 22, 2020, and is hereby incorporated by reference in its entirety.

### TECHNICAL FIELD

The present disclosure relates to swine production, in particular breeding, specifically selection of gilt with a higher probability of fertility through vaginal lipidome profiles.

### BACKGROUND

Swine producers rely on relative size and health at weaning to select gilts to replace sows for future breeding. The gilts are selected when they are weaned from the dam, which occurs at approximately three weeks of age. The gilts are then raised as a group until six months of age, at which time the swine producers determine if the gilts exhibit estrus. Those that do are bred using artificial insemination.

A recent study of 350+ gilts demonstrated that as many as 43% were considered infertile since they either did not exhibit estrus or they did but did not become pregnant. This high failure rate is costly in terms of time and money.

If more infertile sows could be identified at a much earlier age, i.e., as gilts around the time of weaning (e.g., around three weeks), swine producers could save a significant amount of time and money. It is an object of the present disclosure to provide such a method. This and other objects and advantages, as well as inventive features, will become apparent from the detailed description provided herein.

### SUMMARY

A method of eliminating a gilt from selection for breeding is provided. The method comprises (i) obtaining a vaginal lipidome profile from a gilt at around the time of weaning and (ii) using the vaginal lipidome profile to assign the gilt as probable fertile or infertile, wherein a decreased level of arachidonic acid (AA), a decreased level of docosahexaenoic acid (DHA), and/or an increased level of very long-chain fatty acids (VLCFA) compared to a normal gilt vaginal lipidome profile is/are indicative of infertility. A gilt's vaginal lipidome profile can comprise a decreased level of AA, a decreased level of DHA, and an increased level of VLCFA compared to a normal gilt vaginal lipidome profile. The VLCFA comprises (i) at least one of cerotic acid and ximenic acid and (ii) optionally at least one of nonadecanoic acid and pentadecanoic acid. The method can be combined with measuring the size of the vulva.

A method of analyzing a lipidome profile of a gilt vaginal swab is also provided. The method comprises (i) receiving a vaginal swab obtained from a gilt at around the time of weaning and (ii) analyzing, or having analyzed, lipids obtained from the vaginal swab for at least one lipid selected from the group consisting of AA, DHA, and VLCFA, and (iii) determining, or having determined, a decreased level of AA, a decreased level of DHA, and/or an increased level of VLCFA in the lipids compared to corresponding levels of a normal gilt vaginal lipidome profile. The VLCFA comprises (i) at least one of cerotic acid and ximenic acid and (ii) optionally at least one of nonadecanoic acid and pentadecanoic acid.

Further provided is a kit for obtaining a vaginal swab from a gilt. The kit comprises (i) materials for cleaning a vulva, (ii) a tool for scraping a surface of an anterior vagina, (iii) a sample container, which can be sealed, for holding the tool after use, and (iv) instructions for obtaining the vaginal swab. The kit can further comprise a mailing container.

### DETAILED DESCRIPTION

The present disclosure is predicated, at least in part, on the discovery that lipid profiles of vaginal swabs of weaning gilts are predictive of infertility. More specifically, the present disclosure is predicated on the discovery that sow infertility is associated with lower levels of arachidonic acid (AA; C20:4) and docosahexaenoic acid (DHA; C22:6) and higher levels of very long-chain fatty acids (VLCFA), specifically cerotic acid (C26:0) and ximenic acid (C26:1).

In view of the above, a method of eliminating a gilt from selection for breeding is provided. The method comprises (i) obtaining a vaginal lipidome profile from a gilt at around the time of weaning (e.g., 21±4 days) and (ii) using the vaginal lipidome profile to assign the gilt as probable fertile or infertile, wherein a decreased level of AA, a decreased level of DHA, and/or an increased level of VLCFA compared to a normal gilt vaginal lipidome profile is/are indicative of infertility. A gilt's vaginal lipidome profile can comprise a decreased level of AA, a decreased level of DHA, and an increased level of VLCFA compared to a normal gilt vaginal lipidome profile. The VLCFA comprises (i) at least one of cerotic acid and ximenic acid and (ii) optionally at least one of nonadecanoic acid and pentadecanoic acid.

A method of analyzing a lipidome profile of a gilt vaginal swab is also provided. The method comprises (i) receiving a vaginal swab obtained from a gilt at around the time of weaning and (ii) analyzing, or having analyzed, lipids obtained from the vaginal swab for at least one lipid selected from the group consisting of AA, DHA, and VLCFA, and (iii) determining, or having determined, a decreased level of AA, a decreased level of DHA, and/or an increased level of VLCFA in the lipids compared to corresponding levels of a normal gilt vaginal lipidome profile. The VLCFA comprises (i) at least one of cerotic acid and ximenic acid and (ii) optionally at least one of nonadecanoic acid and pentadecanoic acid.

Lipids can be extracted and analyzed using any suitable methods as known in the art and exemplified herein. A preferred method of extracting lipids is the Bligh and Dyer method (Canadian J of Biochem and Physiol 37: 911-917 (1959)). A preferred method of analyzing lipids is set forth in Example 4.

Further provided is a kit for obtaining a vaginal swab from a gilt. The kit comprises (i) materials for cleaning a vulva, (ii) a tool for scraping a surface of an anterior vagina, (iii) a sample container, which can be sealed, for holding the tool after use, and (iv) instructions for obtaining the vaginal swab. The kit can further comprise a mailing container.

Any suitable materials can be used for cleaning the vulva. Examples include alcohol, such as ethanol or a solution of ethanol and water, and a disposable wipe, such as a wipe made of natural, e.g., cotton (such as gauze), or synthetic fibers, which are absorbent and preferably lint-free.

Any suitable tool can be used for scraping the surface of the anterior vagina. An example is a human pap smear brush, which is commercially available, such as the Rovers® EndoCervix-Brush® (Oss, Netherlands).

Any suitable container, which desirably can be sealed, can be used for holding the tool after use. A preferred sample container is made from plastic, such as polypropylene. Also preferred is a sample container having a conical shape that can receive liquid and can be placed in a centrifuge.

While any suitable mailing container can be used, desirably the container can maintain the samples at a cool/cold temperature, such as until delivered to a laboratory for lipid extraction and analysis.

### EXAMPLES

The following examples serve to illustrate the present disclosure. The examples are not intended to limit the scope of the claimed invention in any way.

### Example 1

Gilt Selection

Piglets were weaned at 21±4 days. Gilts (1,084) from the initial pool of animals were selected for placement in a farm's onsite nursery. At the time of weaning, gilts were weighed and swabs of the anterior vagina were taken for lipidomic analysis.

At 25 weeks of age, gilts were moved from the nursery to the onsite gilt development unit (GDU). Gilts in the GDU were exposed to boars daily to induce puberty. Gilts were observed daily for signs of estrus, and the dates of first and second estrus were recorded.

When a second estrus was detected, the gilt was bred using artificial insemination on the third estrus. If the gilt did not show any signs of estrus after three weeks, estrus was induced (full dose of PG 600; Intervet America, Inc., Millsboro, DE), and the gilt was bred on the next estrus. If the gilt did not respond to induction, it was culled from the selection pool.

### Example 2

Obtention and Storage of Gilt Anterior Vaginal Swabs

The vulva was sprayed with ethanol and wiped clean with gauze. Using a human pap smear brush (Rovers® EndoCervix-Brush®, Oss, Netherlands), the swab was placed into the vagina as far as possible and rotated clockwise to get a representative scraping of the anterior vagina.

Swabs were taken in duplicate and placed in 15 ml polypropylene conical tubes (Corning™ Falcon™ Corning, NY). The tubes were immediately placed on ice, transported to a lab, and stored at −80° C.

### Example 3

Categorization of Fertility Groups

Data from time of birth were compiled after fertile study animals had at least two farrowings. Animals that were culled from the breeding herd for non-reproductive reasons (e.g., lameness, disease, or leg injury) were removed from the data set. The data set included birth weights, weaning weights, vulva measurements, date of birth, date of first boar exposure, dates of estrus detection, date of mating, cull date, reason for culling, farrowing date, and number of piglets born alive. Out of 400 gilts, data were compiled for 353. The 353 gilts were assigned to one of four reproductive performance categories based on pregnancy rate, pigs per sow per year (PSY), and obtention of a vaginal swab. A subset of animals categorized as highly fertile (HF; n=28) consisted of sows that had at least 26 PSY. The HF subset was compared to a subset of animals categorized as infertile (IF; n=34). The IF subset consisted of gilts that did not show any signs of estrus following boar exposure and did not become pregnant.

### Example 4

Identification of Fertility Lipid Biomarkers

Vaginal swab samples were thawed at room temperature. EndoCervix-Brush® samples were rinsed with 500 μl deionized water to remove vaginal cells from the brush. The rinses were vortexed in their respective 15 ml polypropylene conical tubes to lyse the cellular material.

Lipids were extracted using the Bligh and Dyer method (Canadian J of Biochem and Physiol 37: 911-917 (1959)). A sample (200 μl) of each supernatant was transferred to a 1.7 ml tube (Axygen®, Corning, NY). Methanol (prepared with butylated hydroxytoluene 50 ng/mL; 450 μl) and chloroform (250 μl) were added to the supernatant. The sample was vortexed and incubated for 15 min at 4° C. Deionized water (250 μl) and chloroform (250 μl) were added. The sample was then centrifuged at 3,000 rcf at 4° C. for 10 min. The solution was separated into three phases—metabolite, protein, and lipid. The lipid phase was removed, placed in a 1.7 ml microcentrifuge tube, and dried in vacuum concentrator for 8 hrs. The dried pellet was resuspended in 3:6.65:0.35 acetonitrile, methanol and ammonium acetate (200 μl). A 10× solution of sample in solvent was used for analysis.

Multiple reaction monitoring (MRM) profiling was done using a two-step process, beginning with a discovery phase followed by a screening phase. The discovery phase was used to determine which lipid classes were present in each phenotype. Samples (10 μl from each) were pooled by phenotype into a 1.7 ml tube and dried under nitrogen for 8 hrs.

Dried lipid extracts were diluted in 200 μl of 3:6.65:0.35 of acetonitrile, methanol and ammonium acetate. Each pooled sample (8 μl) was injected into a micro-autosampler (G1377A) in a QQQ6410 triple quadrupole mass spectrometer (Agilent Technologies, San Jose, CA) equipped with an ESI ion source. A solvent solution containing acetonitrile with 1% formic acid at 10 μl/min was pumped between injections (CapPump G1376A, Agilent Technologies, San Jose, CA). A solution containing a mixture of methanol and chloroform was injected between samples to remove any remaining lipids from the previous injection.

In the discovery phase, pooled samples were screened for specific chemical classes including acylcarnitines (AC), cholesteryl esters (CE), ceramides, free fatty acids (FFA), phosphatidylcholines (PC), phosphatidylethanolamine (PE), phosphatidylinositols (PI), phosphatidylglycerols (PG), phosphatidylserines (PS), triacylglycerols (TAG), and lipids that reflect different postnatal diets (Harlow et al., PloS one 14: e0215186 (2019)). Vaginal lipids that discriminated between gilts that were suckled versus bottle-fed were referred to as “Method 1” (Harlow et al. (2019), supra). Vaginal lipids that discriminated gilts that received fat supplementation versus gilts that did not receive fat supplementation were referred to as “Method 2” (Harlow et al. (2019), supra). The lipids screened in both methods were from the chemical classes indicated above.

The initial chemical class data were completed using MSConvert20, which converted each set of profiling method data into mzML format. Signal intensity for ions present in neutral loss and precursor ion mass spectra was obtained using an inhouse script. Ions with values of counts >30% of the respective blank within each profiling method were selected as parent ions, and the product ion or neutral loss information was used for selecting ion pairs for the screening phase.

The most robust chemical classes identified during the discovery phase were used in the screening phase of each individual gilt's vaginal swab. The samples were screened four times each with different chemical classes included in each evaluation. The chemical classes in the four screens were PC, FFA, Method 1 lipid classes, and Method 2 lipid classes.

The relative intensity of MRM in each sample was calculated. MRM ion pairs with intensities <1.3-fold of blank sample were removed. The relative intensity of MRM ion pairs was calculated by dividing the average intensity of all lipids with a sample by screening analysis. The relative intensities of MRM ion pairs were uploaded into MetaboAnalyst 4.0 and data were normalized using autoscaling. T-test analysis was used to identify MRMs that distinguished between fertility phenotypes, using an alpha of 0.05 of nominal p-value to identify differentially distributed lipids. Biomarker analysis was completed using classical univariate receiver operating characteristic (ROC) curve analysis with area-under-the-curve (AUC) value used to determine a lipid's potential as a biomarker. Lipids were scored as potential biomarkers using the AUC scale of 0.9-1.0 (excellent), 0.8-0.9 (good), 0.7-0.8 (fair), 0.6-0.7 (poor), and 0.5-0.6 (fail).

The discovery phase identified 269 unique MRMs in pooled samples from HF and IF vaginal swabs. PC accounted for 30% of the unique MRMs identified, followed by FFA, which accounted for 13%.

The screening phase identified 6/36 FFA lipids that differed between HF and IF gilts. Arachidonic acid (AA) and docosahexaenoic acid (DHA) were higher in HF than IF gilts, whereas very long-chain fatty acids (VLCFA), specifically cerotic acid, ximenic acid, nonadecanoic acid, and pentadecanoic acid, were lower in HF than IF gilts. Linear regression analysis indicated that AA was negatively related (p<0.05) to cerotic acid (r=−0.57), ximenic acid (r=−0.35), and nonadecanoic acid (r=−0.49) across both groups. Regression analysis with fertility groups found stronger relationships between AA and cerotic acid and ximenic acid with IF gilts.

Thus, the data indicate a link between infertility and low levels of AA and DHA and higher levels of VLCFA in neonatal vaginal tissue.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

What is claimed is:

1. A method of eliminating a gilt from selection for breeding, which method comprises:
(i) obtaining a vaginal lipidome profile from a gilt at around the time of weaning and
(ii) using the vaginal lipidome profile to assign the gilt as probable fertile or infertile, wherein:
a decreased level of arachidonic acid (AA), a decreased level of docosahexaenoic acid (DHA), and/or an increased level of very long-chain fatty acids (VLCFA) compared to a normal gilt vaginal lipidome profile is/are indicative of infertility,
whereupon a gilt is eliminated from selection for breeding.

2. The method of claim 1, wherein the gilt's vaginal lipidome profile comprises a decreased level of AA, a decreased level of DHA, and an increased level of VLCFA compared to a normal gilt vaginal lipidome profile.

3. The method of claim 1, wherein the VLCFA comprises (i) at least one of cerotic acid and ximenic acid and (ii) optionally at least one of nonadecanoic acid and pentadecanoic acid.

4. The method of claim 2, wherein the VLCFA comprises (i) at least one of cerotic acid and ximenic acid and (ii) optionally at least one of nonadecanoic acid and pentadecanoic acid.

5. A method of analyzing a lipidome profile of a gilt vaginal swab, which method comprises:
(i) receiving a vaginal swab obtained from a gilt at around the time of weaning and
(ii) analyzing, or having analyzed, lipids obtained from the vaginal swab for at least one lipid selected from the group consisting of arachidonic acid (AA), docosahexaenoic acid (DHA), and very long-chain fatty acids (VLCFA), and
(iii) determining, or having determined, a decreased level of AA, a decreased level of DHA, and/or an increased level of VLCFA in the lipids compared to corresponding levels of a normal gilt vaginal lipidome profile,
whereupon a lipidome profile of a gilt vaginal swab is analyzed.

6. The method of claim 5, wherein the VLCFA comprises (i) at least one of cerotic acid and ximenic acid and (ii) optionally at least one of nonadecanoic acid and pentadecanoic acid.

7. A kit for obtaining a vaginal swab from a gilt, which kit comprises:
(i) materials for cleaning a vulva,
(ii) a tool for scraping a surface of an anterior vagina,
(iii) a sample container, which can be sealed, for holding the tool after use, and
(iv) instructions for obtaining the vaginal swab.

8. The kit of claim 7, which further comprises a mailing container.
